(19) United States
(12) Patent Application Publication
(10) Pub. No.: US 2005/0026280 A1
(43) Pub. Date: Feb. 3, 2005

(54) Gene involved in chroloplast RNA-editing

(76) Inventors: Masao Tasaka, Ikoma-shi (JP); Toshiharu Shikanai, Ikoma-shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/740,618

(22) Filed: Dec. 22, 2003

(30) Foreign Application Priority Data
Jul. 18, 2003 (JP) 2003-199098

(51) Int. Cl. C07H 21/04; C12N 15/82; C12N 15/87; C12N 15/09; C12N 15/63; C12N 15/29

(52) U.S. Cl. 435/410; 536/23.6; 435/320.1; 435/468

(57) ABSTRACT

An Arabidopsis thaliana mutant crr4 having a mutation in at least one base of the CRR4 genomic gene shown in SEQ ID NO. 1, which is obtained by treating an Arabidopsis thaliana (wild type) with a mutagen, preparing plants of the next generation of the mutagen-treated Arabidopsis thaliana (wild type), and selecting a plant whose NDH activity has been decreased, as compared with that of Arabidopsis thaliana (wild type), from the plants of the next generation.

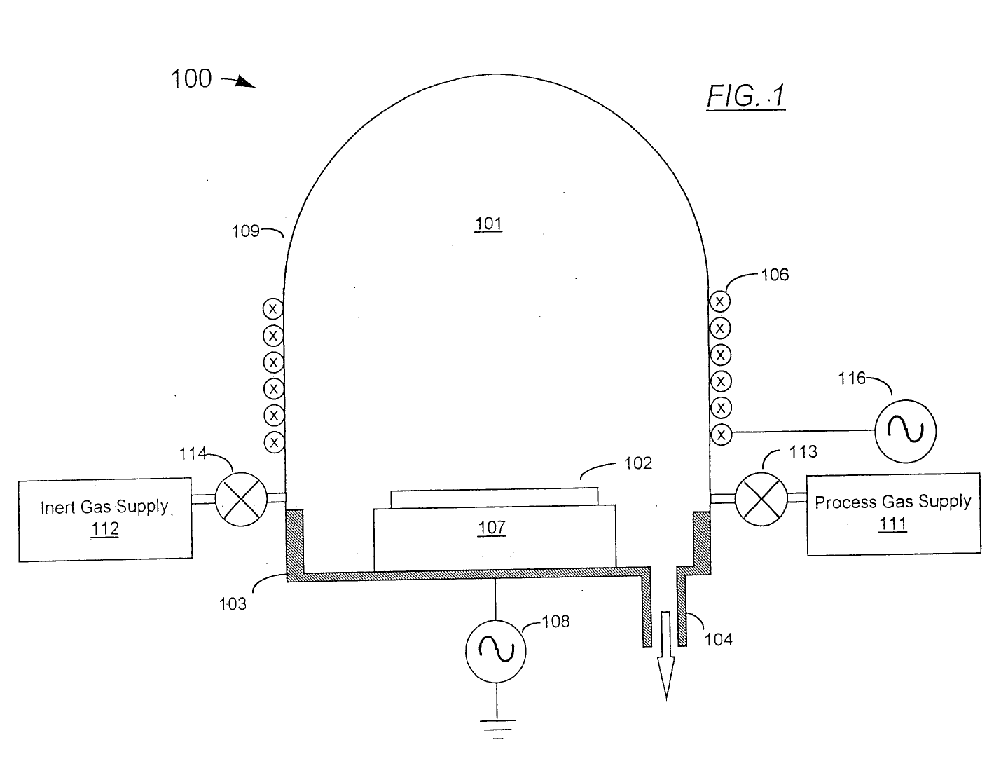

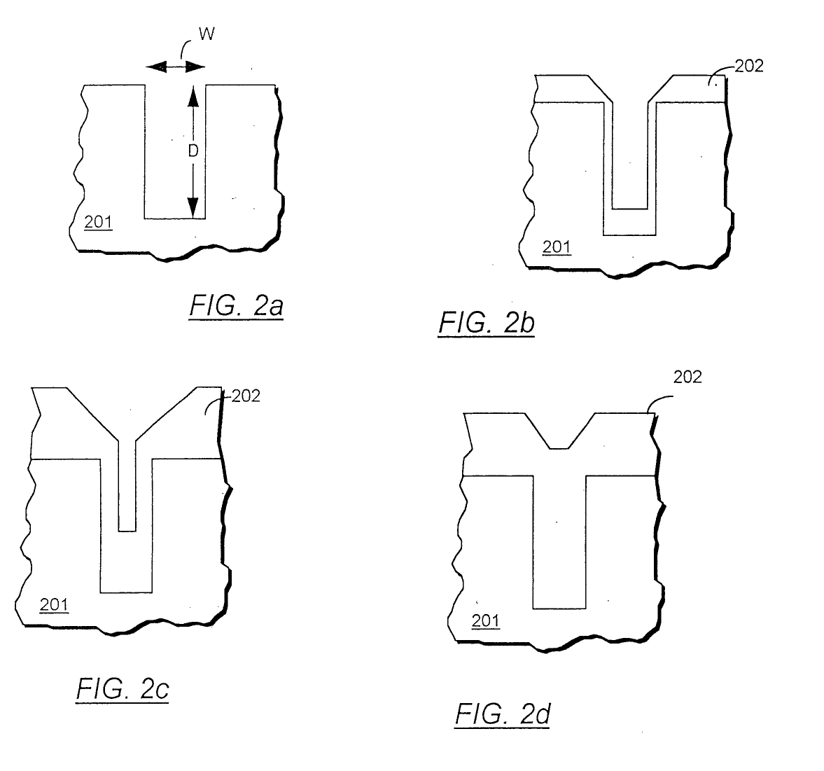

### CROSS-REFERENCE TO RELATED APPLICATIONS

This application is based upon and claims the benefit of priority from the prior Japanese Patent Application No. 2003-199098, filed Jul. 18, 2003, the entire contents of which are incorporated herein by reference.

### BACKGROUND OF THE INVENTION

1. Field of the Invention

The present invention relates to a factor involved in a function of RNA-editing modifying genetic information encoded in genome after transcription. Specifically, the present invention relates to a protein involved in a function of RNA-editing and a gene encoding the protein. As the factor of the present invention is capable of modifying genetic information expressed by transcription, without causing modification of genome, it is expected that RNA-editing will be made possible in an artificially controllable manner, by using the factor.

2. Description of the Related Art

Plants carry out photosynthesis by utilizing light energy. For plants, light is essential for photosynthesis. However, when plants absorb an excess amount of light, harmful active oxygen is generated, which causes light-resulting damage to the plant. In order to adapt to fluctuation of light intensity, plants have various mechanisms for recognizing light intensity and adjusting efficiency of light absorption. Specifically, plants have mechanisms for maximizing photosynthetic activity thereof under weak light, and decreasing, on the contrary, the photosynthetic activity and safely discarding excessive light energy under strong light. More specifically, it has been reported that the wild-type strain of Arabidopsis thaliana adjusts the efficiency of light absorption in response to irradiation of strong light, thereby avoiding reception of excessive light energy.

There has been a report on a factor which is involved with the above-described mechanisms by the use of a mutant whose mechanisms for avoiding reception of excessive light energy has been made dysfunctional. Genetic analysis clarified genes involved in the mechanisms adjusting the efficiency of light energy utilization. Specifically, there have been reports on NPQ1 (non-photochemical quenching 1) and NPQ4 as factors inside chloroplast, which prevent generation of active oxygen by safely discarding excessive light energy (refer to Reference 1 and Reference 2 described below). Further, as a factor which controls the function of these factors inside chloroplast, there have been reported PGR5 (Reference 3) and chloroplast NAD(P)H dehydrogenase (which will be referred to as “chloroplast NDH” or “NDH” hereinafter) (Reference 4).

It has been known, from a knockout of a tobacco gene utilizing chloroplast transformation, that chloroplast NDH is involved in cyclic electron transport around photosystem I. This electron transport forms a proton gradient through thylakoid membrane between the stroma and lumen. It has been reported that, acidification of thylakoid lumen causes a reaction of avoiding reception of excessive light energy by way of NPQ1 and NPQ4.

Reference 1: Niyogi, K.K., Grossman, A. R., and Bjorkman, O. (1998). “Arabidopsis mutants define a central role for the xanthophyll cycle in the regulation of photosynthetic energy conversion.” Plant Cell 10, 1121-1134.

Reference 2: Li, X.-P., Bjorkman, O., Shih, C., Grossman, A. R., Rosenquist, M., Jannsson, S., and Niyogi, K.K. (2000). “A pigment-binding protein essential for regulation of photosynthetic light harvesting.” Nature 403, 391-395.

Reference 3: Munekage, Y., Hojo, M., Meurer, J., Endo, T., Tasaka, M., and Shikanai, T. (2002) “PGR5 is involved in cyclic electron flow around photosystem I and is essential for photoprotection in Arabidopsis.” Cell 110, 361-371.

Reference 4: Shikanai, T., Endo, T., Hashimoto, T., Yamada, Y., Asada, K., and Yokota, A. (1998) “Directed disruption of the tobacco ndhB gene impairs cyclic electron flow around photosystem I.” Proc. Natl. Acad. Sci. USA 95, 9705-9709.

### BRIEF SUMMARY OF THE INVENTION

One object of the present invention is to provide a mutant which lacks the activity of chloroplast NDH, by using the activity of chloroplast NDH as an index. Another object of the present invention is to identify a gene which causes mutation to the mutant and thereby provide the gene, and to provide a protein having a specific function, which is encoded by the gene.

The present inventors have produced a mutant which lacks the activity of chloroplast NDH, by using the activity of chloroplast NDH as an index, and identified a gene which causes mutation to the mutant. The present inventors have also newly revealed that the gene encodes “a factor which is involved in editing of RNA encoding a subunit of chloroplast NDH”, and completed the present invention. Specifically, the present invention provides the following means.

(1) An Arabidopsis thaliana mutant crr4 having a mutation in at least one base of the CRR4 genomic gene shown in SEQ ID NO: 1, which is obtained by treating an Arabidopsis thaliana (wild type) with a mutagen; preparing plants of the next generation of the mutagen-treated Arabidopsis thaliana (wild type); and selecting a plant whose NDH activity has been decreased, as compared with that of Arabidopsis thaliana (wild type), from the plants of the next generation.

(2) An Arabidopsis thaliana mutant crr4, whose NDH activity has been decreased, as compared with that of Arabidopsis thaliana (wild type), due to a mutation of at least one base of the CRR4 genomic gene shown in SEQ ID NO: 1 in Arabidopsis thaliana (wild type).

(3) An Arabidopsis thaliana mutant crr4, whose NDH activity has been decreased, as compared with that of Arabidopsis thaliana (wild type), due to a missense mutation of at least one base of the CRR4 genomic gene shown in SEQ ID NO: 1 in Arabidopsis thaliana (wild type).

(4) An Arabidopsis thaliana mutant crr4, whose NDH activity has been decreased, as compared with that of Arabidopsis thaliana (wild type), due to a nonsense mutation of at least one base of the CRR4 genomic gene shown in SEQ ID NO: 1 in Arabidopsis thaliana (wild type).

(5) An Arabidopsis thaliana mutant crr4, whose NDH activity has been decreased, as compared with that of Arabidopsis thaliana (wild type), due to a mutation of the CRR4 genomic gene shown in SEQ ID NO: 1 in Arabidopsis thaliana (wild type), wherein the mutation is selected from the group consisting of the following (A) to (D):

(A) a mutation in which the 1273th base “G” of the CRR4 genomic gene shown in SEQ ID NO: 1 has been substituted with “A”;
(B) a mutation in which the 1190th base “C” of the CRR4 genomic gene shown in SEQ ID NO: 1 has been substituted with “T”;
(C) a mutation in which the 1235th base “G” of the CRR4 genomic gene shown in SEQ ID NO: 1 has been substituted with “A”; and

(D) a mutation in which the 1384th base “G” of the CRR4 genomic gene shown in SEQ ID NO: 1 has been substituted with “A”.

(6) A protein selected from the group consisting of the following (a) and (b):
(a) a protein comprising the amino acid sequence of SEQ ID NO: 2 and involved in an RNA-editing function; and
(b) a protein comprising an amino acid sequence of SEQ ID NO: 2, in which one or a few amino acids of the amino acid sequence have been deleted, substituted and/or added and which is involved in an RNA-editing function.

(7) A gene encoding a protein selected from the group consisting of the following (a) and (b):
(a) a protein comprising the amino acid sequence of SEQ ID NO: 2 and involved in an RNA-editing function; and
(b) a protein comprising an amino acid sequence of SEQ ID NO: 2, in which one or a few amino acids of the amino acid sequence have been deleted, substituted and/or added and which is involved in an RNA-editing function.

(8) A gene selected from the group consisting of the following (c) or (d):
(c) a gene comprising the nucleotide sequence of SEQ ID NO: 1 and encoding a protein involved in an RNA-editing function; and
(d) a gene comprising a nucleotide sequence of SEQ ID NO: 1, in which one or a few bases of the nucleotide sequence have been deleted, substituted and/or added and which encodes a protein involved in an RNA-editing function.

(9) A recombinant vector, comprising the gene described in the aforementioned (7) or (8).

(10) A transformant, comprising the gene described in the aforementioned (7) or (8).

As described above, the present invention provides an Arabidopsis thaliana mutant crr4, which lacks the NDH activity. The present invention has revealed for the first time that the protein encoded by the gene which causes mutation to the mutant crr4 is involved in a function of RNA-editing. Accordingly, the present invention provides a protein involved in a function of RNA-editing, as well as a gene encoding the protein.

It is expected that artificial control of RNA-editing becomes possible by developing the study of RNA-editing by utilizing the mutant and the gene of the present invention. Further, in the long run, it is expected that artificial modification of expressed genetic information can be carried out after transcription, by RNA-editing, so that such modification can be used for specifically killing or damaging cancer cells, or treating genetic diseases.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### DETAILED DESCRIPTION OF THE INVENTION

The present invention will be described in detail hereinbelow. It should be noted that the following descriptions are provided only for illustrating the present invention and do not restrict the present invention.

[Mutant of Arabidopsis thaliana]

The Arabidopsis thaliana mutant crr4 of the present invention (which will be also referred to as “mutant crr4” hereinafter) is a mutant having a mutation in at least one base (e.g., one or a few bases) of the CRR4 genomic gene shown in SEQ ID NO: 1, which is obtained by treating an Arabidopsis thaliana (wild type) with a mutagen; preparing plants of the next generation of the mutagen-treated Arabidopsis thaliana (wild type); and selecting a plant whose NDH activity has been decreased, as compared with that of Arabidopsis thaliana (wild type), from the plants of the next generation.

In another aspect of the present invention, the mutant crr4 of the present invention is a mutant, whose NDH activity has been decreased, as compared with that of Arabidopsis thaliana (wild type), due to a mutation of at least one base (e.g., one or a few bases) of the CRR4 genomic gene shown in SEQ ID NO: 1 in Arabidopsis thaliana (wild type). It should be noted that the mutant crr4 of the present invention has the same characteristics as the wild type, except that the mutant crr4 exhibits low NDH activity.

In the present invention, “a mutation” represents any mutation, as long as the mutation causes a decrease in the normal amount of the chloroplast NDH complex which would naturally be possessed by Arabidopsis thaliana (wild type) and thus lowers the NDH activity of the plant. For example, the mutation may represent substitution, deletion or addition of at least one base. More specifically, the mutation may include missense mutation and nonsense mutation. The “missense mutation” is a mutation in which at least one base (e.g., one or a few bases) of the CRR4 genomic gene is substituted with a base(s) of different type(s), whereby a codon designating an amino acid is substituted with a codon designating an amino acid of another type. On the other hand, the “nonsense mutation” is a mutation in which at least one base (e.g., one or a few bases) of the CRR4 genomic gene is substituted with a base(s) of different type(s), whereby a codon designating an amino acid is substituted with a termination codon.

Examples of the mutant crr4, which were actually produced in the present invention, include a mutant of Arabidopsis thaliana, whose NDH activity has been decreased, as compared with that of Arabidopsis thaliana (wild type), due to a mutation of the CRR4 genomic gene shown in SEQ ID NO: 1 in Arabidopsis thaliana (wild type), wherein the mutation is selected from the group consisting of the following (A) to (D).

(A) A mutation in which the 1273th base “G” of the CRR4 genomic gene shown in SEQ ID NO: 1 has been substituted with “A”. Due to this mutation, “glycine” as the 425th amino acid of the CRR4 protein shown in SEQ ID NO: 2 is substituted with “arginine”.

(B) A mutation in which the 1190th base “C” of the CRR4 genomic gene shown in SEQ ID NO: 1 has been substituted with “T”. Due to this mutation, “alanine” as the 397th amino acid of the CRR4 protein shown in SEQ ID NO: 2 is substituted with “valine”.

(C) A mutation in which the 1235th base “G” of the CRR4 genomic gene shown in SEQ ID NO: 1 has been substituted with “A”. Due to this mutation, “tryptophan” as the 412th amino acid of the CRR4 protein shown in SEQ ID NO: 2 is substituted with “the termination codon”.

(D) A mutation in which the 1384th base “G” of the CRR4 genomic gene shown in SEQ ID NO: 1 has been substituted with “A”. Due to this mutation, “glutamic acid” as the 462th amino acid of the CRR4 protein shown in SEQ ID NO: 2 is substituted with “lysine”.

Hereinafter, the mutants having mutations of the aforementioned (A) to (D) will be referred to as the mutants crr4-1, crr4-2, crr4-3 and crr4-4, respectively.

The seed of Arabidopsis thaliana (wild type) (which will be also referred to as “wild type” hereinafter), which is used for producing the mutant crr4 of the present invention, are available from Lehle seeds Co., Ltd. (Round Rock Tex. 78681 USA, http://www.arabidopsis.com/). As Arabidopsis thaliana (wild type), the seed which is called “Columbia g11” (which includes “g11” as a marker so that seeds of other types are prevented from being mixed therewith) can be used. Alternatively, seeds of the wild type which have been subjected to a mutagen treatment (i.e., ethylmethane sulfonic acid treatment) can also be purchased from the aforementioned seed company. Arabidopsis thaliana (wild type) exhibits normal NDH activity. However, as the substrate (the electron donor) of NDH is not known at the time of filing the present application, it is not possible to quantitatively express the NDH activity.

In the production of the mutant crr4 of the present invention, Arabidopsis thaliana (wild type) is first subjected to a mutagen treatment. As the wild type to be subjected to the mutagen treatment, seed, plant body, callus and the like may be used. In the present invention, techniques commonly known as a mutagen treatment may be used. Examples of the mutagen include a chemical mutagen such as an alkylating agent which alkylates a base of DNA; an electromagnetic wave which causes damages to DNA such as ultraviolet rays and X-rays; and a radioactive substance. Alternatively, the mutagen treatment may be carried out according to the known Agrobacterium infection method, in which a DNA region interposed between a pair of border sequences (25-base sequences) located at both ends of T-DNA region of Ti plasmid contained in Agrobacterium is inserted into a random site of the genome DNA of the wild type. Preferably, the mutagen treatment is carried out by immersing seeds of the wild type in a solution containing 0.2 to 0.3 wt % of a chemical mutagen (e.g., ethylmethane sulfonic acid) for 12 to 16 hours. In a case in which seeds are used as the wild type, the seeds of wild type are each grown to plants.

Next, the mutagen-treated wild type (the plant body) is made to perform self-pollination, whereby the next generation is produced. Among the thus produced next generation, a plant whose NDH activity has been decreased, as compared with that of the wild type (i.e., a plant having a mutation caused by the mutagen treatment in a homozygous form) is selected. Here, the expression that “the NDH activity has been decreased, as compared with that of the wild type” means that, when the NDH activity of the wild type is expressed as 100%, the NDH activity of the mutant corresponds to 0 to 10% thereof and, preferably, to 0% (i.e., the mutant completely lacks the NDH activity). It should be noted that, as it is difficult to quantitatively express the NDH activity, as described above, the NDH activity described in the present invention represents an approximate degree of activity estimated from a change in chlorophyll fluorescence and an accumulated amount of the subunit proteins, each of which varies in accordance with the NDH activity.

With regard to the details of the method of measuring the NDH activity, the descriptions of examples described below may be referred to.

The plant selected at this stage is a mutant whose NDH activity has been decreased, as compared with the wild type, and can be regarded as the “mutant crr4” of the present invention. In the present invention, four types of lines (crr4-1, crr4-2, crr4-3 and crr4-4) have been selected as the mutant crr4. It has been found that all of the four types of lines are plants having a mutation inside the region of the same gene (which will be also referred to as “CRR4 genomic gene” hereinafter).

The mutations observed in the aforementioned four types of lines are recessive and inherited according to Mendelian inheritance. Therefore, the mutations are not mutations that have been generated in the gene of NDH subunit encoded in the chloroplast genome, but mutations that have been generated in the genomic gene. In addition, when the CRR4 gene of the wild type is introduced to the mutant crr4 of the present invention, the NDH activity of the mutant crr4 recovers to the normal level. Therefore, it is concluded that the aforementioned mutations are mutations that have been generated in the CRR4 genomic gene (refer to example 3 described below).

As described above, the production of the mutant crr4 of the present invention is reproducible. That is, the mutagen-treated wild-type Arabidopsis thaliana is made to perform self-pollination. Among the thus obtained next generation, a plant whose NDH activity has been lowered, as compared with that of the wild type, is selected. Then, it is confirmed that the selected plant has a mutation in the nucleotide sequence of the CRR4 genomic gene.

Once the mutant crr4 is produced, the mutant crr4 can be reproduced by performing self-pollination of the mutant crr4.

The mutant crr4 of the present invention is useful when the mechanisms of RNA-editing is studied in the molecular level.

[CRR4 Gene and Protein Encoded by CRR4 Gene]

In the present invention, the gene (CRR4 gene) which had caused the mutations in the mutant crr4 was identified, as described in example 2 mentioned below.

The nucleotide sequence of the CRR4 genomic gene is shown in SEQ ID NO: 1, and the amino acid sequence of the protein encoded by the CRR4 genomic gene is shown in SEQ ID NO: 2. The nucleotide sequence shown in SEQ ID NO: 1 and the amino acid sequence shown in SEQ ID NO: 2 are those which do not have any mutation therein.

The present invention has revealed that, in Arabidopsis thaliana (wild type), when the CRR4 genomic gene has a mutation, maturation of RNA of chloroplast gene ndhD (i.e., production of the translation-initiation codon by RNA-editing) is disturbed (refer to example 4). Accordingly, it was found by the present invention that the protein encoded by the CRR4 genomic gene functions when mRNA of chloroplast gene ndhD is edited and thereby the translation-initiation codon is produced. More specifically, it has been revealed that the CRR4 protein recognizes the sequence of a specific RNA of chloroplast and is involved in RNA-editing in which a specific base cytosine is substituted with uracil. Further, it has also been revealed that the CRR4 gene of the present invention encodes members of PCMP family which are presumably involved in maturation of RNA in chloroplast.

Accordingly, the present invention provides a gene comprising the nucleotide sequence shown in SEQ ID NO: 1 and encoding a protein involved in an RNA-editing function. In this gene, one or a few bases in the nucleotide sequence shown in SEQ ID NO: 1 may be deleted, substituted and/or added, as long as the gene encodes a protein having capability to be involved in an RNA-editing function.

Further, the present invention provides a gene which encodes the following protein: a protein comprising the amino acid sequence shown in SEQ ID NO: 2 and involved in an RNA-editing function. In this gene, one or a few amino acids in the amino acid sequence of the aforementioned protein may be deleted, substituted and/or added, as long as the gene encodes a protein having capability to be involved in an RNA-editing function.

Yet further, the present invention provides a protein comprising the amino acid sequence shown in SEQ ID NO: 2 and involved in an RNA-editing function. In this protein, one or a few amino acids in the amino acid sequence of SEQ ID NO: 2 may be deleted, substituted and/or added, as long as the protein has capability to be involved in an RNA-editing function.

[Recombinant Vector Containing CRR4 Gene and Transformant Containing CRR4 Gene]

1. Recombinant Vector

A recombinant vector containing CRR4 gene of the present invention can be obtained by inserting CRR4 gene of the present invention to an appropriate vector. The vector to which CRR4 gene of the present invention is inserted is not particularly limited, as long as the vector enables replication in a host. Examples thereof include plasmid DNA, phage DNA and the like. Specific examples of plasmid DNA include a plasmid for Escherichia coli such as pBR322, pBR325, pUC118 and pUC119; a plasmid for Bacillus subtilis such as pUB110 and pTP5; a plasmid for yeast such as YEp13, YEp24 and YCp50; and a plasmid for a plant cell such as pBI221 and pBI121. Specific examples of phage DNA include λ phage and the like. Alternatively, animal virus vector such as retrovirus and vaccinia virus; insect virus vector such as baculovirus; and plant virus vector may be used as a vector. When the CRR4 gene of the present invention is inserted into a vector, there is employed a method including, for example, the steps of: cleaving the cloned CRR4 gene by treatment with an appropriate restriction enzyme; inserting the CRR4 gene into a restriction enzyme site or a multi-cloning site of an appropriate vector DNA and thereby connecting the CRR4 gene to the vector. It is necessary that the CRR4 gene of the present invention is incorporated to the vector such that the function of the gene can be fully effected. Therefore, the vector of the present invention may optionally contain cis element such as an enhancer, a splicing signal, a poly(A)-addition signal, a selective marker, ribosome binding sequence (SD sequence) or the like, as well as a promoter and the CRR4 gene of the present invention. Examples of the selective marker include the dihydrofolate reductase gene, the ampicillin-resistant gene and the neomycin-resistant gene.

Specifically, the recombinant vector of the present invention can be prepared by inserting the CRR4 gene of the present invention to binary vector such as pBI101, under the control of the constitutive cauliflower mosaic virus ³⁵S promoter incorporated within the binary vector.

2. Transformant

The portion of a plant, as the object of the transformation in the present invention, may be any of the following: a plant as a whole; organs of the plant (such as leaf, petal, stem, root and seed); plant tissues (such as epidermis, phloem, parenchyma, xylem and vascular bundle); and cultured cells of the plant. Plants of any type may generally be used for transformation. Examples thereof include monocotyledons such as rice, corn, asparagus and wheat and dicotyledons such as Arabidopsis thaliana, tobacco, carrot, soybean, tomato and potato.

Any appropriate conventional method known in the art may be employed as a method of producing the transformant of the present invention. For example, the aforementioned recombinant vector may be introduced to a plant by the conventional transformation method such as electroporation method, Agrobacterium method, particle gun method, PEG method or the like.

In a case in which a plant tissue, a plant organ or cultured cells are used as the object of the transformation, the tissue, the organ or the cultured cells can be regenerated to a plant, by administering plant hormones (such as auxin, cytokinin, gibberellin, abscisic acid, ethylene and brassinolide) at appropriate concentrations, according to the conventional plant tissue culture method.

A transformant of the present invention can be obtained, not only by introducing the CRR4 gene of the present invention to the aforementioned plant host, but also by introducing the CRR4 gene to a host including bacteria such as Escherichia coli, yeast, animal cells or insect cells, without being restricted to such examples. When bacteria such as Escherichia coli or yeast is used as a host, the recombinant vector of the present invention preferably contains a sequence enabling autonomous replication in the host, a promoter, ribosome binding sequence, the gene of the present invention and the transcription termination sequence. The recombinant vector may further include a sequence which regulates the promoter.

Whether the CRR4 gene of the present invention has been incorporated to the host or not can be confirmed by PCR method, Southern hybridization method, Northern hybridization method or the like. For example, in the PCR method, DNA is extracted as a template for PCR from the transformant, primers specific to the CRR4 gene are designed, and PCR is carried out. PCR can be carried out in the substantially same condition as in the preparation of the aforementioned plasmid. Thereafter, the PCR product obtained as a result of the amplification is subjected to agarose gel electrophoresis, polyacrylamide gel electrophoresis or capillary electrophoresis, and dyed by treatment with ethidium bromide, SYBR Green or the like. As a result, the amplified PCR product is detected as a single band, and thereby it can be confirmed that the transformation is successful. Alternatively, a primer labeled in advance with fluorescence dye or the like may be used in PCR, so that the amplified PCR product can be detected from fluorescence. Or, the amplified PCR product may be bound to the solid phase of a microplate or the like, and detected from fluorescence or enzymatic reactions.

### EXAMPLES

The present invention will be described in detail by the following examples. The present invention is not restricted by the following descriptions.

### Example 1

### Production of Mutant crr4 having Recessive Mutation of CRR4 Gene

About 6000 seeds of Arabidopsis thaliana (wild type) were immersed in 0.3 wt % ethylmethane sulfonic acid (EMS) solution for 16 hours, whereby the mutagen treatment of Arabidopsis thaliana (wild type) was carried out. The mutagen-treated seeds (M1 seeds) were each grown to plants, and self-pollination of the grown plant was performed, thereby preparing the next generation. Among the next generation (50,000 plants), plants having decreased chloroplast NDH activity were selected. Specifically, the chloroplast NDH activity was measured according to the method described below.

Seedling of Arabidopsis thaliana is left in darkness for at least 15 minutes. Thereafter, white light of 100 μmol photons m⁻²·sec⁻¹ is irradiated thereon for 5 minutes. A transient increase in chlorophyll fluorescence, which occurs in 1 to 2 minutes immediately after the irradiation of white light, is observed by using a CCD camera or PAM chlorophyll fluorometry. The transient increase in chlorophyll fluorescence represents the NDH activity.

Plants whose measured NDH activity was, when the NDH activity of the wild type is expressed as 100, 10 or less (i.e., plants in which chlorophyll fluorescence was hardly observed) were selected. Then, it was confirmed that the characteristic possessed by the selected plant (i.e., low NDH activity) is stably inherited to the next generation. After the confirmation, the selected plant was identified as “mutant crr4” of the present invention and named “mutant crr4”. In the present example, four lines of mutant crr4 (crr4-1, crr4-2, crr4-3 and crr4-4) were selected.

Further, it was confirmed that the genome DNA of the mutant crr4 had a mutation in CRR4 gene (shown in SEQ ID NO: 1), by analyzing the nucleotide sequence thereof. This confirmation was carried out by amplifying the genome DNA region including the CRR4 gene of the mutant crr4 by using a pair of primers shown below. That is, the genome DNA region including the CRR4 gene of the mutant crr4 was amplified by PCR, the entire nucleotide sequence of the PCR product was determined, and the determined entire nucleotide sequence was compared with the nucleotide sequence of the CRR4 gene of the wild-type Arabidopsis thaliana.

| | |
|---|---|
| 5′-ATAAACCCAATCCGGTTCATC-3′ | (SEQ ID NO: 3) |
| 5′-GCCAAGGAATTGCAGAATAGG-3′ | (SEQ ID NO: 4) |

From the comparison, it was confirmed that substitution of amino acid of CRR4 protein, due to missense mutation of CRR4 genomic gene, had occurred in the mutants crr4-1, crr4-2 and crr4-4. Further, it was confirmed that deletion of the C terminal sequence of CRR4 protein, due to nonsense mutation of CRR4 genomic gene, had occurred in the mutant crr4-3. In all of the aforementioned four lines, the function of CRR4 protein had been lowered or lost due to such mutations as described above.

### Example 2

### Isolation of CRR4 Gene

The CRR4 gene which causes the mutation in each mutant crr4 was isolated and identified by positional cloning.

Specifically, the mutant crr4-1 (ecotype; Columbia) was crossed with the wild type (ecotype; Landsberg erecta), whereby F1 generation was obtained. Then, F2 generation as the next generation of F1 generation was prepared by performing self-pollination of the F1 generation. Among the F2 generation, plants which lacked the NDH activity were selected.

The chromosomes of the selected plants were analyzed by using molecular markers. That is, the molecular markers are PCR primers, and the nucleotide sequence of the PCR product amplified by the molecular markers distinguishes the case where the amplified chromosome region is derived from Columbia from the case where it is derived from Landsberg erecta. In other words, the PCR product amplified by the molecular markers differs between the case where the amplified chromosome region is derived from Columbia and the case where it is derived from Landsberg erecta. Based on such difference of the PCR product, each chromosome region of the selected plants were analyzed. The crr4 mutant gene always exists in Columbia-derived chromosome region, because ecotype of the mutant crr4-1 is Columbia. Therefore, the region that was specified as Columbia-derived chromosome region in common with all of the selected plants was identified as the crr4 gene locus.

Further, for each of mutants crr4-1 to crr4-4, the identified region (i.e., the region identified as the crr4 gene locus) was searched for a gene having the chloroplast transport signal. The nucleotide sequence of the gene having the chloroplast transport signal was determined. The determined nucleotide sequence of the gene was compared with the nucleotide sequence of the wild type gene, whereby the mutation-causing CRR4 gene was isolated and identified.

### Example 3

### Experiment in which NDH Activity was Recovered in Mutant crr4

In this example, the wild-type CRR4 gene was introduced to the mutant crr4-1 and the mutant crr4-2. As a result, the NDH activity of the mutants crr4-1 and crr4-2 was recovered.

At first, cloning of the wild-type CRR4 gene was carried out as follows. A DNA fragment including the wild-type CRR4 gene was prepared by PCR using a pair of primers: 5′-CGCTCTTTTCACAACCTATGC-3′ (SEQ ID NO: 5) and 5′-CATGAATTCTCAAACCAAAGACC-3′ (SEQ ID NO: 6). The prepared PCR product was cleaved by treatment with XbaI and EcoRI, and it was inserted to a vector pBIN19, thereby the wild-type CRR4 gene was cloned. As a result, the nucleotide sequence interposed between the sequence of TCTAGACATGATTACATATT (SEQ ID NO: 7) and the sequence of CTCAAACCAAAGACCATCCA (SEQ ID NO: 8) was cloned.

As described above, the 3.8 kb of genome DNA fragment of Arabidopsis thaliana interposed between the sequence of TCTAGACATGATTACATATT (SEQ ID NO: 7) and the sequence of CTCAAACCAAAGACCATCCA (SEQ ID NO: 8) was cloned in the vector pBIN19. The 3.8 kb of genome DNA fragment includes the wild-type CRR4 genomic gene, the regions ranging 1.7 kb-upstream and the regions ranging 0.3 kb-downstream therefrom. The mutants crr4-1 and crr4-2 were transformed, respectively, with the vector containing the wild-type CRR4 genomic gene by way of infection of Agrobacterium M90. The transformants were selected by utilizing kanamycin resistance as index. The transformants having resistance to kanamycin exhibited recovery of NDH activity.

Further, when another vector which expresses the wile-type CRR4 gene under control of cauliflower mosaic virus ³⁵S promoter was introduced to the mutants crr4-1 and crr4-2, the NDH activity in the transformants was recovered.

From the results described above, it was proved that the mutant of the present invention has a mutation in the CRR4 gene.

### Example 4

### Experiment in which Involvement of CRR4 Protein in RNA-Editing was Demonstrated

In the present invention, it has been revealed that the CRR4 gene encodes members of PCMP family which is presumably involved in maturation of RNA in chloroplast. The mutant crr4 specifically lacks the chloroplast NDH activity. It is assumed that maturation of 11 of chloroplast RNAs which encode the subunit of NDH has been damaged in the mutant crr4.

RNA was extracted from each of the mutants crr4-1 to crr4-4, and Northern analysis of the extracted RNA was carried out by using the 11 NDH subunit genes as a probe. No difference was found between the NDH subunit genes of the mutants crr4-1 to crr4-4 and the NDH subunit genes of the wild type. For this reason, the RNA sequence of ndhB, ndhD and ndhF genes (i.e., genes encoding the subunit of the NDH complex), RNA-editing thereof having been reported, were examined by directly sequencing the RT-PCR products thereof.

The following primers were used in order to amplify each RNA obtained as a transcription product of ndhB, ndhD and ndhF genes (i.e., genes encoding the subunit of the NDH complex).

| | |
|---|---|
| (5′ side of ndhB gene): | |
| 5′-TTTGCTTCTCTTCGATGGAAG-3′ | (SEQ ID NO: 9) |
| and | |
| 5′-ACGACTGGAGTGGGAGATCCTTC-3′; | (SEQ ID NO: 10) |
| (3′ side of ndhB gene): | |
| 5′-CGTATACGAAGGATCTCCCAC-3′ | (SEQ ID NO: 11) |
| and | |
| 5′-CCTGAGCAATCGCAATAATCG-3′; | (SEQ ID NO: 12) |
| (ndhD gene): | |
| 5′-TTGAGTACGCGTTCTTTGGAC-3′ | (SEQ ID NO: 13) |
| and | |
| 5′-AATAGCTCCATTAAGTCCAGG-3′; | (SEQ ID NO: 14) |
| (ndhF gene): | |
| 5′-ACCTATTTTACTAGGAGTTGGAC-3′ | (SEQ ID NO: 15) |
| and | |
| 5′-AGCATTCGCTGCAATAGGTCG-3′. | (SEQ ID NO: 16) |

From the obtained results, it was revealed that, in all of the mutants crr4-1 to crr4-4, only the RNA-editing for producing the translation-initiation codon of ndhD gene had completely been inhibited.

The RNA sequence that is obtained by transcription of the coding region of ndhD gene and RNA-editing in the wild type, is shown in SEQ ID NO: 17. In the RNA sequence of SEQ ID NO: 17, the second uracil (U) is a base obtained by substituting the original cytosine (C) with RNA-editing. On the contrary, in the mutant crr4 of the present invention, such RNA-editing did not occur, so that the original cytosine (C) remained at the same position without being substituted with uracil (U).

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An Arabidopsis thaliana mutant crr4 having a mutation in at least one base of the CRR4 genomic gene shown in SEQ ID NO: 1, which is obtained by treating an Arabidopsis thaliana (wild type) with a mutagen;
preparing plants of the next generation of the mutagen-treated Arabidopsis thaliana (wild type); and selecting a plant whose NDH activity has been decreased, as compared with that of Arabidopsis thaliana (wild type), from the plants of the next generation.

2. An Arabidopsis thaliana mutant crr4, whose NDH activity has been decreased, as compared with that of Arabidopsis thaliana (wild type), due to a mutation of at least one base of the CRR4 genomic gene shown in SEQ ID NO: 1 in Arabidopsis thaliana (wild type).

3. An Arabidopsis thaliana mutant crr4, whose NDH activity has been decreased, as compared with that of Arabidopsis thaliana (wild type), due to a missense mutation of at least one base of the CRR4 genomic gene shown in SEQ ID NO: 1 in Arabidopsis thaliana (wild type).

4. An Arabidopsis thaliana mutant crr4, whose NDH activity has been decreased, as compared with that of Arabidopsis thaliana (wild type), due to a nonsense mutation of at least one base of the CRR4 genomic gene shown in SEQ ID NO: 1 in Arabidopsis thaliana (wild type).

5. An Arabidopsis thaliana mutant crr4, whose NDH activity has been decreased, as compared with that of Arabidopsis thaliana (wild type), due to a mutation of the CRR4 genomic gene shown in SEQ ID NO: 1 in Arabidopsis thaliana (wild type), wherein the mutation is selected from the group consisting of the following (A) to (D):
(A) a mutation in which the 1273th base “G” of the CRR4 genomic gene shown in SEQ ID NO: 1 has been substituted with “A”;
(B) a mutation in which the 1190th base “C” of the CRR4 genomic gene shown in SEQ ID NO: 1 has been substituted with “T”;
(C) a mutation in which the 1235th base “G” of the CRR4 genomic gene shown in SEQ ID NO: 1 has been substituted with “A”; and
(D) a mutation in which the 1384th base “G” of the CRR4 genomic gene shown in SEQ ID NO: 1 has been substituted with “A”.

6. A protein selected from the group consisting of the following (a) and (b):
(a) a protein comprising the amino acid sequence of SEQ ID NO: 2 and involved in an RNA-editing function; and
(b) a protein comprising an amino acid sequence of SEQ ID NO: 2, in which one or a few amino acids of the amino acid sequence have been deleted, substituted and/or added and which has capability to be involved in an RNA-editing function.

7. A gene encoding a protein selected from the group consisting of the following (a) and (b):
(a) a protein comprising the amino acid sequence of SEQ ID NO: 2 and involved in an RNA-editing function; and
(b) a protein comprising an amino acid sequence of SEQ ID NO: 2, in which one or a few amino acids of the amino acid sequence have been deleted, substituted and/or added and which has capability to be involved in an RNA-editing function.

8. A gene selected from the group consisting of the following (c) or (d):
(c) a gene comprising the nucleotide sequence of SEQ ID NO: 1 and encoding a protein involved in an RNA-editing function; and
(d) a gene comprising a nucleotide sequence of SEQ ID NO: 1, in which one or a few bases of the nucleotide sequence have been deleted, substituted and/or added and which encodes a protein having capability to be involved in an RNA-editing function.

9. A recombinant vector, comprising the gene according to claim 7.

10. A recombinant vector, comprising the gene according to claim 8.

11. A transformant, comprising the gene according to claim 7.

12. A transformant, comprising the gene according to claim 8.


SEQUENCE LISTING (17 sequences)

SEQ ID NO: 1 - DNA, 1821 bp, Arabidopsis thaliana
  CDS (1)..(1818)
atggagtgttcgatttcatccaccattcatgtccttggaagctgcaaaacttcagatgacgtgaaccaaatccacgggcgattgattaagacgggaatcatcaaaaactcaaatctcactacgaggattgttctggcttttgcctcttctcgacgtccgtatctcgccgatttcgcgcgttgtgtcttccacgagtatcacgtatgttcgttttcatttggagaggtggaggatccatttttatggaacgccgtgatcaagtctcactctcatggaaaggatccgagacaagctctgctcttgctctgtttgatgctcgagaatggggtttccgtggacaaattctcactgtcacttgttcttaaagcgtgttcgaggttaggttttgtaaaaggaggaatgcagattcatgggtttttgaaaaaaactggactttggtcggatttgtttctacagaattgtttgattggcttgtatctgaaatgtggttgtttaggtttatcacgccagatgtttgatagaatgccgaagagagactctgtttcttataattccatgattgatgggtatgtcaaatgtggattgattgtatccgcgcgtgaattgttcgatttgatgcctatggagatgaagaatttgatatcttggaactctatgataagtggttatgctcagacatcagatggagttgacatagcgtctaaactgtttgctgatatgcctgagaaggacttgatttcgtggaactcaatgatagatggatatgtaaaacacggaagaatcgaagatgctaagggtttattcgatgtgatgccgagaagagatgtagttacttgggctaccatgattgatgggtatgcaaagttaggttttgttcatcacgctaagactctgtttgaccaaatgcctcatagagatgttgtggcatataattctatgatggctggttatgttcaaaacaagtatcacatggaagctcttgaaatatttagtgacatggaaaaggagagtcacttgttacccgatgatacgactttggtaatagttctgcctgcaattgctcagcttggccgattatccaaagccatagatatgcatttgtacatcgtggagaaacaattctatctaggtggaaaactcggtgttgctctcattgatatgtattcgaaatgcggaagcatacaacacgccatgttggttttcgagggaatcgaaaacaaaagcattgatcactggaatgctatgattggtgggctcgctattcatggtctaggggaatctgcattcgatatgctcttgcagattgagagactctctttaaaaccagacgatatcacctttgttggcgttttaaatgcttgcagccactctgggttagtaaaggaaggccttctctgctttgagctcatgaggagaaaacacaagatagaaccaagattgcaacactatggttgtatggtagacatactatcgagatccggaagtatagagctagccaaaaacttaatagaggaaatgcctgttgagccaaatgatgtcatatggagaacgtttctcaccgcttgtagtcaccacaaggagtttgaaacgggagagcttgtcgcaaaacaccttattttgcaggctggatataacccgagctcatatgtgctactctctaacatgtatgctagttttggaatgtggaaggatgttcgtagagttagaacgatgatgaaggaaagaaagatagagaaaattcctggttgtagttggattgagctcgatggaagagtccatgagttctttgtagatagcattgaagtttccagtacattgtag

SEQ ID NO: 2 - protein, 606 aa, Arabidopsis thaliana
MECSISSTIHVLGSCKTSDDVNQIHGRLIKTGIIKNSNLTTRIVLAFASSRRPYLADFARCVFHEYHVCSFSFGEVEDPFLWNAVIKSHSHGKDPRQALLLLCLMLENGVSVDKFSLSLVLKACSRLGFVKGGMQIHGFLKKTGLWSDLFLQNCLIGLYLKCGCLGLSRQMFDRMPKRDSVSYNSMIDGYVKCGLIVSARELFDLMPMEMKNLISWNSMISGYAQTSDGVDIASKLFADMPEKDLISWNSMIDGYVKHGRIEDAKGLFDVMPRRDVVTWATMIDGYAKLGFVHHAKTLFDQMPHRDVVAYNSMMAGYVQNKYHMEALEIFSDMEKESHLLPDDTTLVIVLPAIAQLGRLSKAIDMHLYIVEKQFYLGGKLGVALIDMYSKCGSIQHAMLVFEGIENKSIDHWNAMIGGLAIHGLGESAFDMLLQIERLSLKPDDITFVGVLNACSHSGLVKEGLLCFELMRRKHKIEPRLQHYGCMVDILSRSGSIELAKNLIEEMPVEPNDVIWRTFLTACSHHKEFETGELVAKHLILQAGYNPSSYVLLSNMYASFGMWKDVRRVRTMMKERKIEKIPGCSWIELDGRVHEFFVDSIEVSSTL

SEQ ID NO: 3 - DNA, 21 bp, Artificial Sequence
  synthetic oligonucleotide
ataaacccaatccggttcatc

SEQ ID NO: 4 - DNA, 21 bp, Artificial Sequence
  synthetic oligonucleotide
gccaaggaattgcagaatagg

SEQ ID NO: 5 - DNA, 21 bp, Artificial Sequence
  synthetic oligonucleotide
cgctcttttcacaacctatgc

SEQ ID NO: 6 - DNA, 23 bp, Artificial Sequence
  synthetic oligonucleotide
catgaattctcaaaccaaagacc

SEQ ID NO: 7 - DNA, 20 bp, Arabidopsis thaliana
tctagacatgattacatatt

SEQ ID NO: 8 - DNA, 20 bp, Arabidopsis thaliana
ctcaaaccaaagaccatcca

SEQ ID NO: 9 - DNA, 21 bp, Artificial Sequence
  synthetic oligonucleotide
tttgcttctcttcgatggaag

SEQ ID NO: 10 - DNA, 23 bp, Artificial Sequence
  synthetic oligonucleotide
acgactggagtgggagatccttc

SEQ ID NO: 11 - DNA, 21 bp, Artificial Sequence
  synthetic oligonucleotide
cgtatacgaaggatctcccac

SEQ ID NO: 12 - DNA, 21 bp, Artificial Sequence
  synthetic oligonucleotide
cctgagcaatcgcaataatcg

SEQ ID NO: 13 - DNA, 21 bp, Artificial Sequence
  synthetic oligonucleotide
ttgagtacgcgttctttggac

SEQ ID NO: 14 - DNA, 21 bp, Artificial Sequence
  synthetic oligonucleotide
aatagctccattaagtccagg

SEQ ID NO: 15 - DNA, 23 bp, Artificial Sequence
  synthetic oligonucleotide
acctattttactaggagttggac

SEQ ID NO: 16 - DNA, 21 bp, Artificial Sequence
  synthetic oligonucleotide
agcattcgctgcaataggtcg

SEQ ID NO: 17 - RNA, 1503 nt, Arabidopsis thaliana
augaaugauuuuccuugguuaacaauaauuguuguuuuuccaauaucugcugguucauuaauguuauuucucccgcauaggggaaauaaagucaauaaaugguauacuauaugcauuugcaucuuagaacuucuucuaacgaccuacgcuuuuuguuauaauuuuaaaauggacgauccauuaauucaacuguccgaagauuauaaauggaucgauuuuuuugauuuuuacuggagaaugggaauagauggacuuucuauaggaacuauuuuacugacgggauuuauuacuacuuuagccacuuuagcggcuuuuccaguuacucgggauucccgauuuuuucauuuccugauguuagcaauguacagcggucaaauaggauuauuuucuucucgggaucuucuacuuuuuuucaucaugugggaauuagaauuaauucccguuuaucuacuuuuauccaugugggguggaaagaaacgucuguauucggcuacaaaauuuauuuuguacacggcaggaaguucuauuuuuuuauuaauaggaguuuuagguauaaguuuauaugguucgaacgaaccaacauuaaauuuagaacucuuagcuaauaaauccuauccuguuacacuugaaauacuauuuuauauuggauuucuuauugcuuuugccgucaaauuaccgauuauaccuuuacauacuugguuaccugacacccacggugaggcacauuacaguaccuguaugcuucucgcuggaaucuuauuaaaaaugggagccuauggguugguucgaaucaauauggaauuauuaccccacgcucauucuauguuuucuccuugguuguugguagucgguacaauccaaauaauuuaugcagcuuuaacaucucucggucaacguaauuuaaaaaagagaauagccuauucuucuguaucucauauggguuuuauaauuauagguauuaguucuauaacggauccuggacuuaauggagcuauuuuacaaauaauuucucauggauuuauuggcgcugcacuuuuuuucuuggcaggaacgaguuaugauagaauuaggcuuguuuaucuugaugaaauggguggaauggcuaucuccauuccaaaaauauuuacaauguucacuauuuuaucgauggcuucccuugcauugccgggcaugagugguuuuauugcagaauucauuguuuuuuuuggaauaauuaccagccaaaaauauuucuuaauuuccaaaauuuucauuauuuuuguaauggcaauuggaaugauauuaacuccuauauauuuauuaucuauguuacgccaaauguucuauggauacaaguuaauuaauaucaaaaacuuuucuuuuuuugauucuggaccccgagaguuauuucuuucaaucucuauucuuuuacccauaauugguauugggauuuauccugauuuugugcucucauuagcaagugacaaggucgaauccauuuuaucuaauuauuuuuauggauag